(19) United States
(12) Patent Application Publication
(10) Pub. No.: US 2026/0245205 A1
(43) Pub. Date: Aug. 20, 2026

(54) NON-CONTACT, OPTICAL IMAGING METHOD FOR DETECTION OF OCCULT BLOOD IN BODILY SAMPLES

(71) Applicant: University of Washington, Seattle, WA (US)

(72) Inventors: Babak Nazer, Seattle, WA (US); Steve Jacques, Seattle, WA (US)

(73) Assignee: University of Washington, Seattle, WA (US)

(21) Appl. No.: 19/161,143

(22) PCT Filed: Feb. 29, 2024
(86) PCT No.: PCT/US2024/017902

§ 371 Date: Aug. 29, 2025

Related U.S. Application Data
(60) Provisional application No. 63/488,123, filed on Mar. 2, 2023.

(51) Int. Cl. G06T 7/00; G01N 21/31; G01N 33/483; G01N 33/49; G06N 20/00

(52) U.S. Cl. CPC G06T 7/0012; G01N 21/31; G01N 33/4833; G01N 33/49; G06N 20/00; G06T 2207/10036; G06T 2207/30004

(57) ABSTRACT

In some embodiments, a computer-implemented method of detecting a presence or absence of blood in a bodily sample is provided. A computing system receives one or more images of the bodily sample. The computing system determines one or more reflectance characteristics of the bodily sample based on the one or more images of the bodily sample. The computing system determines a presence or absence of blood in the bodily sample based on the one or more reflectance characteristics of the bodily sample within a predetermined wavelength band. The computing system causes an indication of the presence or absence of blood to be presented on a display device.

### CROSS-REFERENCE TO RELATED APPLICATION

This application claims the benefit of Provisional Application No. 63/488,123, filed Mar. 2, 2023, the entire disclosure of which is hereby incorporated by reference herein for all purposes.

### BACKGROUND

Colorectal cancer, or colon cancer, is one of the leading causes of mortality in the US, if not the entire global population. Existing methods for screening colorectal cancer include: a colonoscopy, Guaic-based fecal occult blood test (gFOBT)/fecal immunochemical test (FIT), and Cologuard (or similar stool DNA testing). However, these present screening methods and systems have their limitations. Colonoscopies require patient compliance, a certain level of invasiveness that one would find discomforting, and cost and accessibility, especially if the region in which the patient is located has little to no resources available for medical care. As for gFOBT, FIT, and Cologuard, a patient or user would have to mail in their stool sample to the service provider, creating stigma or wariness for the patient. Furthermore, other gastrointestinal afflictions such as inflammatory bowel disease, Crohn's disease, ulcerative colitis, small bowel angioectasias, and chronic occult gastrointestinal bleeding, are prevalent in our society, and early screening for such affliction is desirable.

There are some approaches to detecting occult blood in bodily samples, such as stool samples. Presently, occult blood is detected visually at approximately 50 mg hemoglobin (Hb)/g stool (mg/g). gFOBT can detect blood at approximately 600 μg Hb/g, and FIT can detect blood at approximately 20 μg Hb/g. However, as mentioned above, the limitations include a user or patient physically handling the stool sample, packaging it, and sending it to their service provider. Furthermore, it may be difficult to assess the presence of blood in a stool sample visually, as many factors can confound the assessment. These include background of the stool sample, amount of occult blood, color of stool sample, presence or absence of ambient light surrounding the stool sample, quality of the device or system capturing the image of the stool sample, and so forth.

Wavelengths of light reflecting off a bodily sample may be used to determine the presence of occult blood. However, present systems and methods fall short as they require additional peripherals or equipment not always available to a user, they use an arbitrary, preset wavelength value or range that is not tailored to the user's profile, and they do not account for the environment and/or background in which the user and bodily sample are located. This can lead to artifacts and noise in the images that can affect the image analysis of the bodily sample, resulting in false positives, or worse yet, false negatives.

There is a need for a system and a method of detecting occult blood in bodily samples that accounts for the environment in which the bodily sample is present, the color of the bodily sample, and/or the background surrounding the bodily sample. Accounting for these factors can lead to more accurate determinations of whether blood is present in the bodily sample.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In some embodiments, a computer-implemented method of detecting a presence or absence of blood in a bodily sample is provided. A computing system receives one or more images of the bodily sample. The computing system determines one or more reflectance characteristics of the bodily sample based on the one or more images of the bodily sample. The computing system determines a presence or absence of blood in the bodily sample based on the one or more reflectance characteristics of the bodily sample within a predetermined wavelength band. The computing system causes an indication of the presence or absence of blood to be presented on a display device. In some embodiments, a non-transitory computer-readable medium is provided having computer-executable instructions stored thereon. The instructions, in response to execution by one or more processors of a computing system, cause the computing system to perform actions of this method. In some embodiments, a computing system is provided that has at least one processor and a non-transitory computer-readable medium having computer-executable instructions stored thereon. These instructions, in response to execution by the at least one processor, cause the computing system to perform actions of this method.

In some embodiments, a computer-implemented method of detecting a presence of blood in a stool is provided. A computing system receives one or more images of stool. The computing system determines one or more reflectance characteristics of the stool based on the one or more images of the stool. The computing system determines a presence or absence of blood in the stool based on the one or more reflectance characteristics of the stool within a predetermined wavelength band. The computing system causes an indication of the presence or absence of blood to be presented on a display device. In some embodiments, a non-transitory computer-readable medium is provided having computer-executable instructions stored thereon. The instructions, in response to execution by one or more processors of a computing system, cause the computing system to perform actions of this method. In some embodiments, a computing system is provided that has at least one processor and a non-transitory computer-readable medium having computer-executable instructions stored thereon. These instructions, in response to execution by the at least one processor, cause the computing system to perform actions of this method.

In some embodiments, a method of training a machine learning model to detect a presence or absence of blood in a bodily sample is provided. A bodily sample spectra is generated based on one or more measured values within a predetermined wavelength band. A false spectra is generated based on one or more interpolated reflectance characteristics between one or more reflectance characteristics at a start point of the predetermined wavelength band and one or more reflectance characteristics at an end point of the predetermined wavelength band. A relationship is extrapolated between the bodily sample spectra and the false spectra, wherein the relationship indicates a presence or absence of blood. In some embodiments, a non-transitory computer-readable medium is provided having computer-executable instructions stored thereon. The instructions, in response to execution by one or more processors of a computing system, cause the computing system to perform actions of this method. In some embodiments, a computing system is provided that has at least one processor and a non-transitory computer-readable medium having computer-executable instructions stored thereon. These instructions, in response to execution by the at least one processor, cause the computing system to perform actions of this method.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIGS. 1A-1B illustrate graphs of measurements of stool samples with no blood added and with 1 mg/g blood added.

FIG. 2A-2F illustrate a non-limiting example embodiment of measuring Red, Green, Blue (RGB) values of stool samples, according to various aspects of the present disclosure.

FIGS. 3A-3B illustrate a non-limiting example embodiment of optical density OD measurements of a stool sample with no blood and with 48 mg/g blood added, according to various aspects of the present disclosure.

FIGS. 4A-4B illustrate a non-limiting example embodiment of Blood Scores depicting levels of blood in stool samples, according to various aspects of the present disclosure.

FIGS. 5A-5B illustrate a non-limiting example embodiment of a ΔOD of stool samples, according to various aspects of the present disclosure.

FIG. 6 illustrates a non-limiting example embodiment of a bodily sample analysis computing system, according to various aspects of the present disclosure.

FIG. 7 illustrates a non-limiting example embodiment of a setup for detecting blood in a bodily sample, according to various aspects of the present disclosure.

FIGS. 8-10 are a flowchart that illustrates a non-limiting example embodiment of a method of detecting a presence or absence of blood in a bodily sample, according to various aspects of the present disclosure.

FIG. 11 is a flowchart that illustrates a non-limiting example embodiment of a method of training a machine learning model to detect a presence or absence of blood in a bodily sample, according to various aspects of the present disclosure.

### DETAILED DESCRIPTION

Given the potential pitfalls of using existing methods and systems of detecting occult blood in a bodily sample, the present disclosure provides a method of detecting occult blood in a bodily sample through non-contact optical imaging. In some embodiments, support for factoring in considerations such as background of the bodily sample, the environment of the bodily sample, and ease for the user or patient are provided.

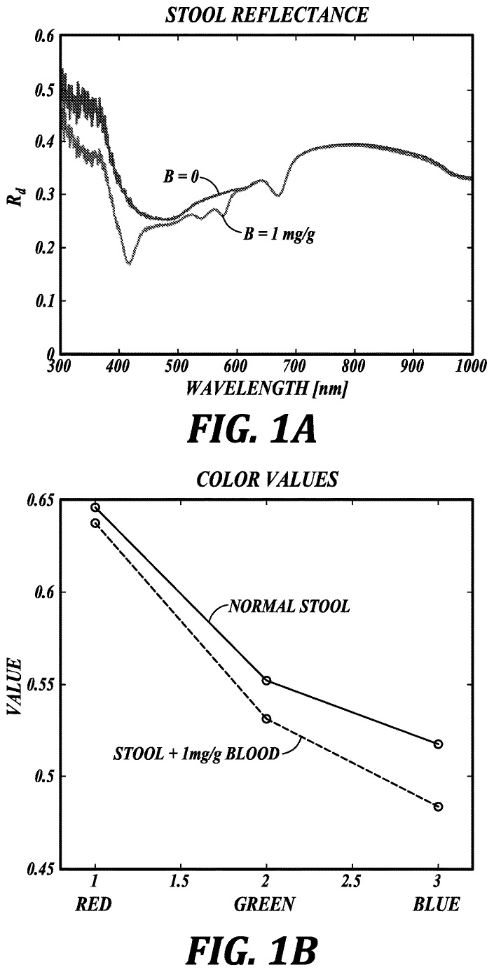

FIG. 1A illustrates a non-limiting example of reflectance (or reflectance spectra) R_{d} before and after 1 mg/g blood was added to a stool sample. From the appearance of this graph, it was determined that the reflectance R_{d} of a stool sample changes when 1 mg/g of blood was added to the stool sample. FIG. 1B illustrates a non-limiting example of measured Red, Green, and Blue (RGB) values of a stool sample before and after 1 mg/g blood was added to the stool sample. Here, it was found that there was a measured difference in RGB values when blood was added to the stool sample, indicating that blood can be measured via RGB quantification.

Further work was undertaken to explore measuring RGB values of blood in stool samples and adjusting the values to improve detectability. An image of a stool sample taken with a camera can be scaled by a scaling constant (or scaling factor) to boost a brightness of the image. In some embodiments, the brightness can be boosted by an amount in a range from 1.5× to 3.5×, such as 2.43×. In some embodiments, boosting the brightness does not change the G/R and/or B/R ratios. In some embodiments, the scaling constant is selected from a range between 1.1× and 2.0×, such as 1.5×. In some embodiments, normalization by RED can be performed to compare a plurality of hues of a plurality of stool samples. In a non-limiting example, two stool samples (sj, bn) were compared, and the mean colors were determined. The results of these determinations are listed in Table 1 (before boosting) and Table 2 (after boosting) below:

TABLE 1
Mean Stool Color Before Boosting

| Color | sj | bn |
|---|---|---|
| RED | 0.433 ± 0.013 | 0.559 ± 0.032 |
| GREEN | 0.345 ± 0.014 | 0.436 ± 0.040 |
| BLUE | 0.298 ± 0.016 | 0.331 ± 0.049 |

TABLE 2
Mean Stool Color After Boosting

| Color | sj | bn |
|---|---|---|
| RED | 1.000 ± 0.000 | 1.000 ± 0.000 |
| GREEN | 0.800 ± 0.012 | 0.769 ± 0.025 |
| BLUE | 0.685 ± 0.023 | 0.581 ± 0.052 |

The above tables indicates that optical measurements of the stool samples changes when blood is added to the stool samples, meaning there is a sensitivity to blood.

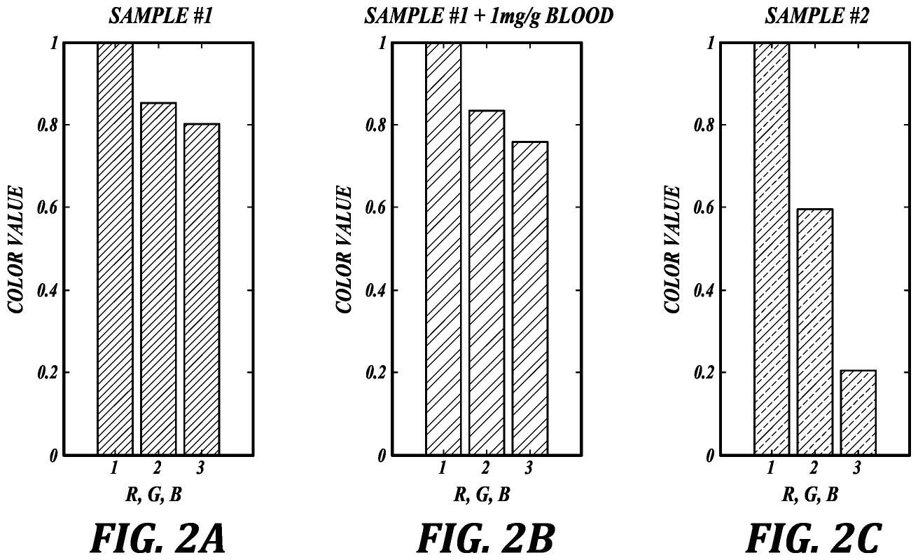

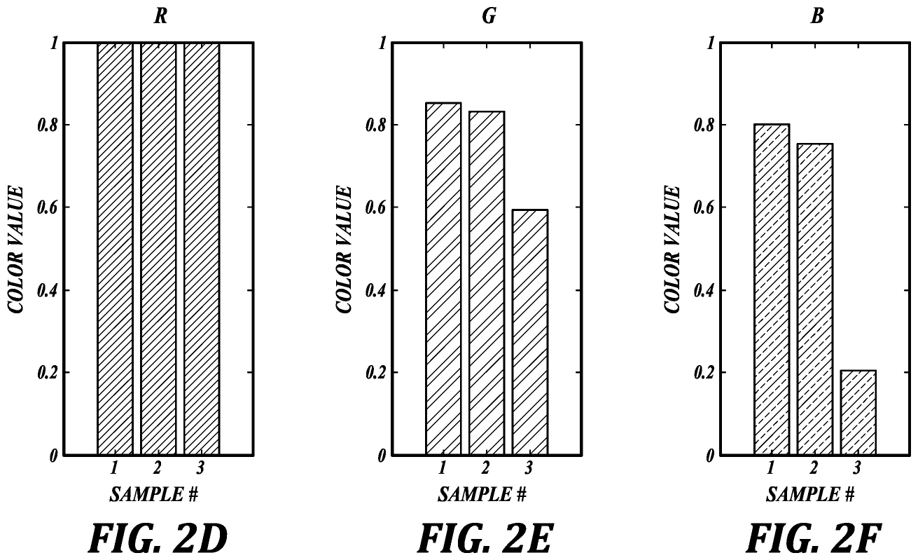

FIGS. 2A-2C illustrate non-limiting examples of bar graphs respectively indicating RGB values of stool sample #1 without blood using calculated spectra, stool sample #1 with blood using calculated spectra, and stool sample #2 (with blood added) taken with a camera. FIGS. 2D-2F illustrate non-limiting examples of bar graphs respectively indicating the R, G, and B color values of stool sample #1 using calculated spectra, stool sample #2 using calculated spectra (which is stool sample #1 with 1 mg/g blood added), and stool sample #3 (which is stool sample #2 taken with a camera). This illustrates that blood in a stool sample provides a measured effect on RGB values, allowing for cameras to determine blood content.

It was found that when measuring reflectance with a spectral collection fiber, the measured reflectance values included artifacts or noise that would affect the measurements when an optical fiber delivered light to and/or from a 5 mm diameter spot. The angle of delivery caused specular reflectance off the air or sample interfaces to reflect away at an angle and not be collected. A reflectance standard is measured for calibration and/or scaling to produce a calibrated reflectance R_{c}. In some embodiments, a reflectance standard in a range of 40-60% is applied. In some embodiments, a 50% reflectance standard is applied. In some embodiments, the reflectance standard is dependent on the specifications of the device used for collecting measurements. In some embodiments, an optical density (or optical density spectra) OD is determined from the calibrated reflectance R_{c}. In some embodiments, the OD is expressed as

$$O D = - {log}_{10} ( {R}_{c} ) \qquad ( 1 )$$

where R_{c} is the calibrated reflectance.

In some embodiments, an iterative step for correcting a measured spectral reflectance R is performed to produce a calibrated reflectance R_{c}. In some embodiments, one or more iterations converts a measured reflectance R into a ratio Nₚ. The ratio Nₚ is expressed as:

$${N}_{p} = \frac{{μ}_{s}^{′}}{{μ}_{a}} = {getN}_{p} ( R ) \qquad ( 2 )$$

where

$${μ}_{s}^{′}$$

represents a reduced scattering, and μₐ represents an absorption coefficient. The absorption coefficient μₐ is expressed as:

$${μ}_{a} = \frac{{μ}_{s}^{′}}{{N}_{p}} \qquad ( 3 )$$

where the above equation is a rearrangement of the previous expression for the ratio Nₚ. The reduced scattering

$${μ}_{s}^{′}$$

is expressed as:

$${μ}_{s}^{′} = a {( \frac{λ}{500 nm} )}^{- b} \qquad ( 4 )$$

where a represents a scattering strength, λ represents a wavelength (nm), and b represents a scattering power. In some embodiments, the scattering strength a is a range between 10-20 cm⁻¹. In some embodiments, the scattering power b is a range between 1-2.

In some embodiments, the reduced scattering

$${μ}_{s}^{′}$$

and the absorption coefficient μₐ are used to generate the calibrated reflectance R_{c}. The expression below is utilized (Farrell T J, M S Patterson, B Wilson, Medical Physics 19:879-888, 1992):

$${R}_{c} = getRdFarrell ( {μ}_{a} , {μ}_{s}^{′} , {n}_{r} ) \qquad ( 5 )$$

where nᵣ is a refractive index ratio. The refractive index ratio nᵣ is expressed as:

$${n}_{r} = \frac{{n}_{stool}}{{n}_{a i r}} \qquad ( 6 )$$

where nₛₜₒₒₗ is a refractive index of the stool sample, and nₐᵢᵣ is the refractive index of air. In some embodiments, the refractive index ratio nᵣ is 1.4.

In some embodiments, each of the one or more iterations causes the measured reflectance R to converge to a newly generated calibrated reflectance R_{c}. In some embodiments, a final absorption coefficient μₐ is generated. The reduced scattering and absorption coefficients combine with nᵣ to yield a reflectance spectrum that matches a measured stool spectrum. A scaling factor K can account for the distance from the stool to the camera, but this does not affect the shape of the spectrum. The optical density versus wavelength allows the difference between (1) the central green wavelengths and (2) the extrapolation of the side wavelengths (blue, red) to the green, to be sensitive to blood (hemoglobin) in the stool. The side wavelengths can account for the background color of the stool.

Through these parameters, it was found that 0.7 mg/g of blood in a stool sample was detectable using light with a wavelength of approximately 410 nm. Furthermore, it was found that 5 mg/g of blood in a stool sample was detectable using light with a wavelength in a range of 550-600 nm. A scattering power b was used in the range of 1-2. A scattering power b of 1 was used in one experiment. A scattering power b of 2 was used in an additional experiment.

In some embodiments, an optical measurement of bodily samples to detect blood is sensitive to about 10 mg/g or more. In some embodiments, blood is seen visually at about 50 mg/g, and so the optical measurement described herein is shown to be more sensitive than visual inspection. Measurements were made over a wavelength range of 400-1000 nm.

Reflectance R and optical density OD of two stool samples (sj=47 g, bn=12 g) were measured, where blood contents B (μg blood/g stool) were added. In some embodiments, 200-48000 μg/g were added to the stool samples. In some embodiments, the reflectance spectra R is expressed as:

$$R = \frac{{M}_{stool} - {M}_{d a r k}}{{M}_{std} - {M}_{d a r k}} {R}_{std} \qquad ( 7 )$$

where M corresponds to measurements (in counts), Mₛₜₒₒₗ corresponds to a measurement of stool with added blood, M_{std} and R_{std} correspond to a 50% reflectance standard (M_{std}, R_{std}=0.50), and M_{dark} corresponds to a measurement with no sample stool. In some embodiments, the reflectance standard (M_{std}, R_{std}=0.50) is utilized as a scaling constant. In some embodiments, the measurement with no sample stool M_{dark} is utilized as a calibration baseline.

An optical density spectra OD of two stool samples (sj=47 g, bn=12 g) was then determined. In some embodiments, an optical density spectra OD (or optical density) is determined in the following expression:

$$OD = - {log}_{10} ( R ) . \qquad ( 8 )$$

In some embodiments, the reflectance characteristic R is expressed as:

$$R \approx a {10}^{- KB} \qquad ( 9 )$$

where a, K are constants unique for each wavelength. The a constant represents a scattering strength (e.g., the reduced scattering of a reference wavelength such as 500 nm), while the K constant is a scaling factor to adjust for the distance from the stool to the camera but does not change the shape of the spectrum. As a result, in some embodiments, the OD is expressed as:

$$OD = KB - lo 710 ( a ) \qquad ( 10 )$$

where OD is linearly related to blood contents B and is utilized as a metric to determine blood content.

In some embodiments, the wavelength at which sensitivity to the presence of blood and/or oxygenated blood is apparent is utilized as the wavelength of interest. In some embodiments, the wavelength range at which blood absorption is present is a predetermined wavelength band. Blood absorption is present within a range of 540-610 nm. 577 nm is a range at which strong absorption by hemoglobin occurs, thereby showing sensitivity to the presence of blood and/or oxygenated blood. 585 nm is presented in situations independent of oxygenated blood. In some embodiments, the range of 500-630 nm is utilized as the predetermined wavelength band. In some embodiments, the 577 nm or the 585 nm is utilized as the wavelength of interest.

A change in R(577 nm) called ΔR is determined, as blood was added to the stool samples. From there, a change in OD(577 nm) called ΔOD was generated using Expression (8). In some embodiments, a background stool absorption is determined using wavelengths neighboring the range at which blood absorption is present. The wavelengths neighboring the 540-610 nm range are 500 nm and 630 nm, where stool absorption outweighs blood absorption. In some embodiments, ΔOD is determined by:

$$Δ OD = 0 D ( 577 nm ) - {OD}_{r e f} ( 577 nm ) \qquad ( 11 )$$

where OD_{ref}(577 nm) is a reference optical density determined by a straight line fit to the optical densities at 500 nm and 630 nm (respectively OD(500 nm) and OD(630 nm)), and then through interpolation. OD(577 nm) is an actual measured optical density of a stool sample. In some embodiments, the ΔOD is utilized as the Blood Score (Bₛ). The Blood Score (Bₛ) is used as an indication of the blood present in the stool sample.

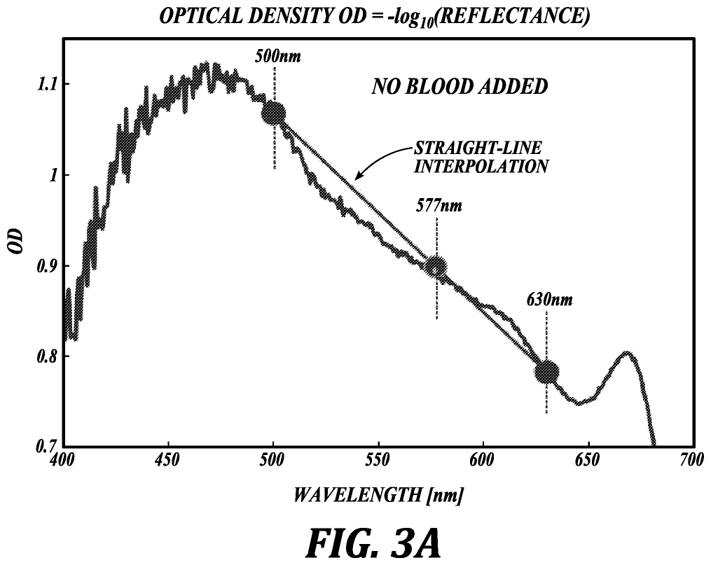

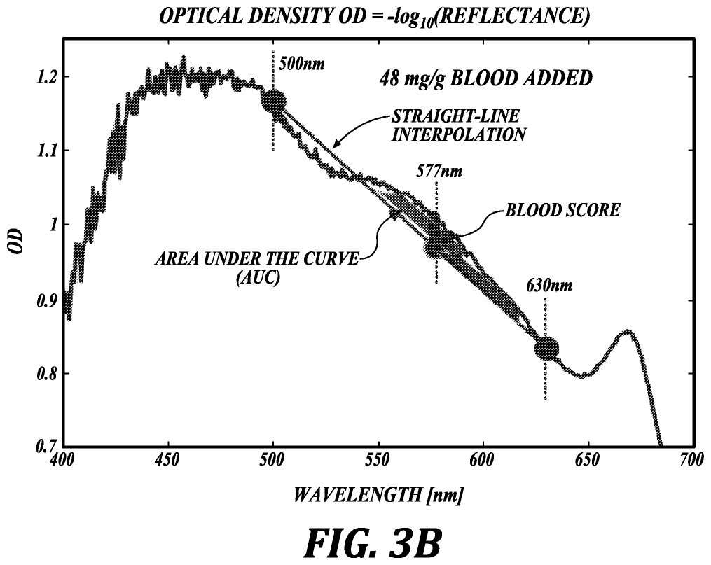

FIGS. 3A-3B respectively illustrate a multispectral, straight-line interpolation analysis of a stool sample (sj) with no blood added, and the sample stool sample with 48 mg/g blood added. A single multispectral measurement was performed using 500 nm, 577 nm, and 630 nm wavelengths. Referring to FIG. 3A, a straight-line interpolation of the 500 nm and 630 nm data yields a reference optical density value at 577 nm (OD_{ref}(577 nm)). Referring to FIG. 3B which is a stool sample with blood, a similar straight-line interpolation is generated using 500 nm and 630 nm to derive OD_{ref}(577 nm), and this is compared to the true optical density OD(577 nm) measured for the stool sample with blood. The solid line (illustrated by the arrow) indicates the difference in optical density and constitutes a Blood Score (Bₛ), as light is reflected back by the blood. In some embodiments, the Blood Score (Bₛ) is calculated by subtracting the OD_{ref}(577 nm) from the OD(577 nm), as shown in Expression (11). In some embodiments, a Blood Score (Bₛ) is determined by measuring a region between the straight-line interpolation and the measured optical density OD to generate an area under the curve (AUC). The AUC is indicated by a shaded region in FIG. 3B.

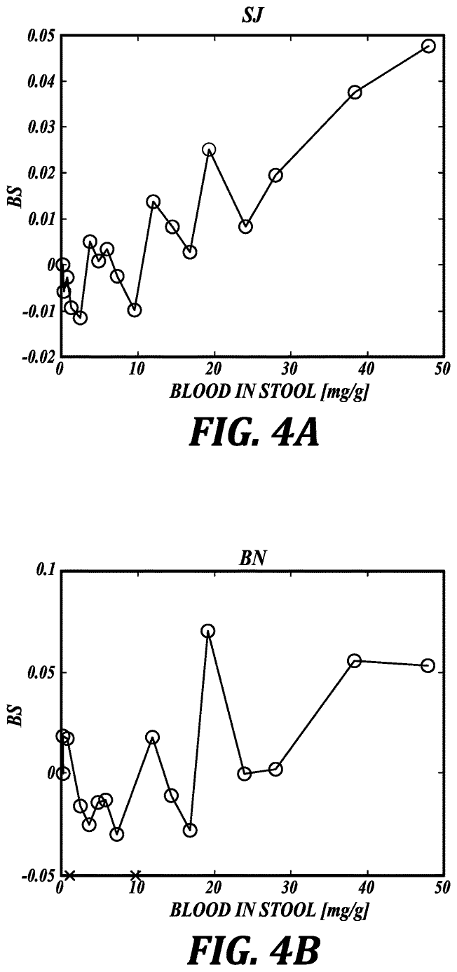

FIGS. 4A-4B illustrate non-limiting examples of analyses of Blood Score (BS) versus added blood to respective stool samples (sj, 47 g) and (bn, 19 g). In some embodiments, the illustrations of FIGS. 4A-4B are utilized as indications of a presence or an absence of blood in a stool or bodily sample. Using the interpolation and AUC analysis determined in FIGS. 3A-3B, as well as Expression (11), a Blood Score (Bₛ) for each stool sample (sj, bn) was calculated and plotted to illustrate the trend of the Blood Score (Bₛ) as blood was added to the stool samples. This indicates that there is an upward trend of the Blood Score (Bₛ) as blood is added using Expression (11) and measured optical density values.

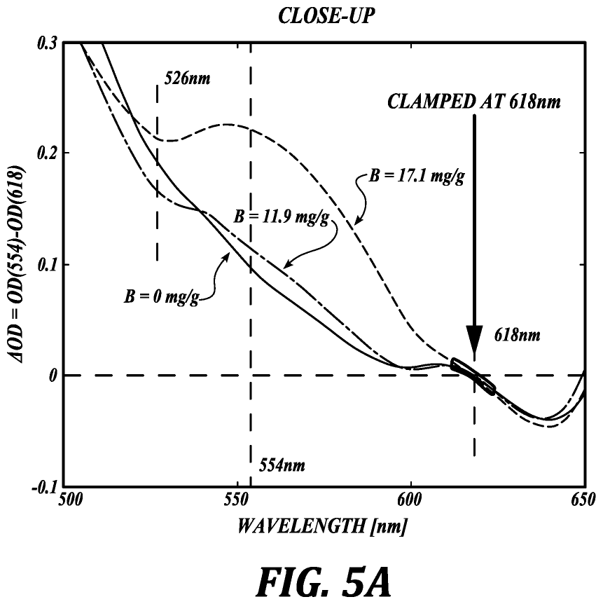

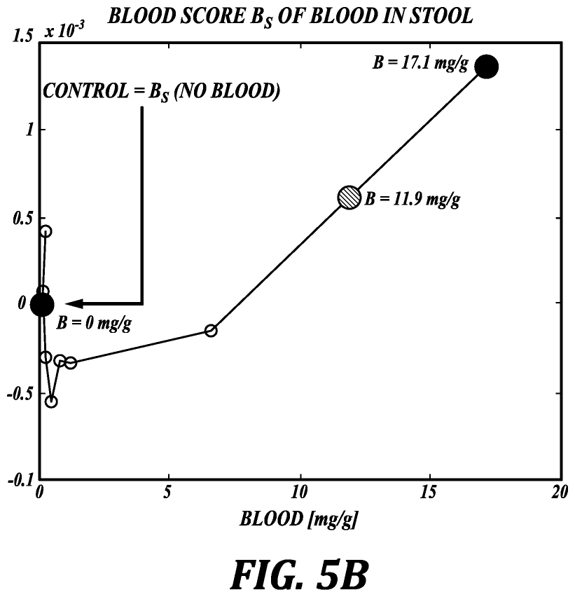

FIGS. 5A-5B illustrate a non-limiting example of an analysis for detecting blood in a stool sample. A raw spectra of a stool sample was obtained, measuring any fluctuations due to the addition of blood to the stool sample. The raw spectra is labeled as M (in counts). An optical reflectance spectra (R_{d}) was acquired. The optical reflectance spectra R_{d} was acquired in a range of 300-1000 nm. The optical reflectance R_{d} is a calibrated optical reflectance spectra R_{d}. In some embodiments, an apparent optical density (OD) is generated based on the optical reflectance spectra R_{d}.

A spectra of a 50% reflectance standard (R_{std}) is utilized. In some embodiments, M₀ includes a measurement with a light source on and a probe or similar measuring device observing a dark and/or empty room. In some embodiments, the M₀ accounts for any light being scattered by imperfections or defects in a sample holder, as most scatter from the air or the sample holder scatters away from the spectral collection fiber.

A region of spectra around a range of 613 nm-623 nm presented a similar optical reflectance standard R_{d} for samples even after the addition of blood. This was used as a reference to account for random fluctuations in the background that offset the signal. A range of the spectra that is responsive to the addition of blood is 526-615 nm. The region of spectra is therefore expressed as 618 nm±5 nm. In some embodiments, the region of spectra that is responsive to blood is utilized as the predetermined wavelength band. The range of 526 nm-615 nm is utilized as the predetermined wavelength band. In some embodiments, the spectra that is responsive to blood is utilized as the wavelength of interest. The 554 nm spectra that is responsive to blood is utilized as the wavelength of interest.

Three ΔOD spectra for three stool samples with amounts of blood B at 0 mg/g, 11.9 mg/g, and 17.1 mg/g were then determined. A cutoff of the spectra is established at 618 nm±5 nm. FIG. 5A illustrates a close-up of the three ΔOD spectra for the three stool samples. Here, ΔOD is expressed as:

$$Δ OD ( B ) = O D ( 554 nm , B ) - OD ( 618 nm \pm 5 nm , B ) . \qquad ( 12 )$$

FIG. 5B illustrates a calculation of a Blood Score Bₛ for increasing amounts of blood B. In some embodiments, the Blood Score Bs is expressed as:

$${B}_{s} = Δ OD ( B ) - Δ OD ( B = 0 ) \qquad ( 13 )$$

where B is the blood content (mg/g) of the stool sample as aliquots of blood were added to the stool sample, and ΔOD(B=0) indicates a difference in ΔOD with no addition of blood to the stool sample.

Through using a spectral measuring device, such as a spectral collection fiber, and through creating mathematical expressions that capture the physical behavior of blood absorbing light, techniques for determining a presence or an absence of blood in bodily samples by measuring wavelengths in RGB images retrieved from cameras have been developed.

While a stool sample was illustrated in the aforementioned examples above, it is foreseeable that the methods and systems described can be applied to various bodily samples, including stool, sputum, urine, lymph fluid, or any fluid-type sample from a human or animal body.

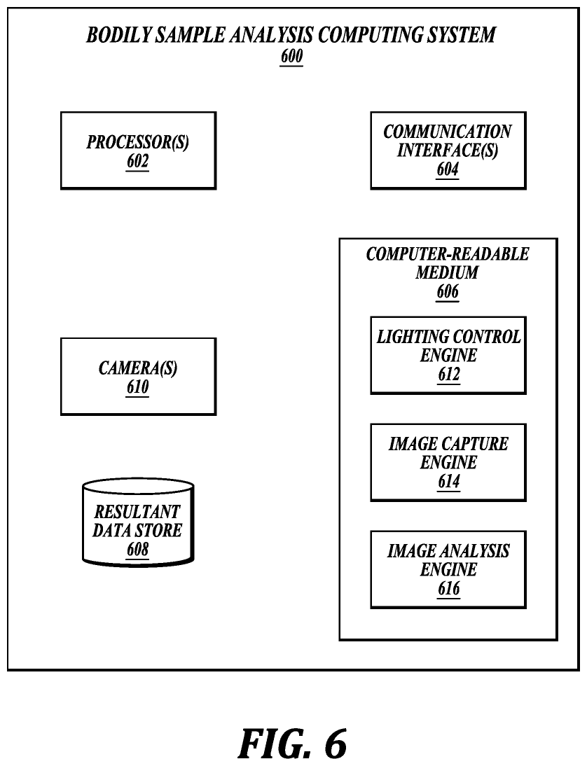

FIG. 6 is a block diagram that illustrates aspects of a non-limiting example embodiment of a bodily sample analysis computing system 600 according to various aspects of the present disclosure. The illustrated bodily sample analysis computing system 600 may be implemented by any computing device or collection of computing devices, including but not limited to a desktop computing device, a laptop computing device, a mobile computing device, a server computing device, a computing device of a cloud computing system, and/or combinations thereof. In some embodiments, at least a portion of the bodily sample analysis computing system 600 may be implemented using a smartphone. In some embodiments, the bodily sample analysis computing system 600 may be communicatively coupled to a plurality of LED lights capable of generating illumination at a variety of wavelengths. In some embodiments, the bodily sample analysis computing system 600 may be communicatively coupled to an illumination source having a single wavelength band. The bodily sample analysis computing system 600 is configured to capture images of bodily samples and to perform analysis/analyses to detect blood therein as described above.

As shown, the bodily sample analysis computing system 600 includes one or more processors 602, one or more communication interfaces 604, one or more cameras 610, a resultant data store 608, and a computer-readable medium 606.

In some embodiments, the one or more processors 602 may include any suitable type of general-purpose computer processor. In some embodiments, the one or more processors 602 may include one or more special-purpose computer processors or AI accelerators optimized for specific computing tasks, including but not limited to graphical processing units (GPUs), vision processing units (VPTs), and tensor processing units (TPUs).

In some embodiments, the one or more communication interfaces 604 include one or more hardware and or software interfaces suitable for providing communication links between components. The one or more communication interfaces 604 may support one or more wired communication technologies (including but not limited to Ethernet, FireWire, and USB), one or more wireless communication technologies (including but not limited to Wi-Fi, WiMAX, Bluetooth, 2G, 3G, 4G, 5G, and LTE), and/or combinations thereof.

In some embodiments, the one or more cameras 610 may include one or more of a spectral camera, a hyperspectral camera, a spectral collection fiber or similar apparatus, a multispectral camera, a spectrometer, or an RGB camera from a user device, such as a webcam, laptop, tablet, smartphone, or similar personal digital assistant (PDA).

As shown, the computer-readable medium 606 has stored thereon logic that, in response to execution by the one or more processors 602, cause the bodily sample analysis computing system 600 to provide a lighting control engine 612, an image capture engine 614, and an image analysis engine 616.

As used herein, “computer-readable medium” refers to a removable or nonremovable device that implements any technology capable of storing information in a volatile or non-volatile manner to be read by a processor of a computing device, including but not limited to: a hard drive; a flash memory; a solid state drive; random-access memory (RAM); read-only memory (ROM); a CD-ROM, a DVD, or other disk storage; a magnetic cassette; a magnetic tape; and a magnetic disk storage.

In some embodiments, the lighting control engine 612 is configured to control an illumination source associated with the bodily sample analysis computing system 600. In embodiments wherein the illumination source is capable of generating illumination at configurable wavelengths, the lighting control engine 612 may be configured to cause LEDs at specific wavelengths to turn on and off, in order to improve the spectral imaging capabilities of the one or more cameras 610 of the bodily sample analysis computing system 600.

In some embodiments, the image capture engine 614 is configured to receive images captured by the one or more cameras 610. In some embodiments, the image capture engine 614 may be configured to perform one or more tasks related to processing the collected images for further analysis, including one or more of associating the images with the wavelength of the LED active during capture, or registering the images with each other such that pixels of the images are aligned with each other. In some embodiments, the image capture engine 614 may capture the images and the lighting control engine 612 may adjust the active LEDs at a very rapid pace, such that the images will already be registered to each other.

In some embodiments, the image analysis engine 616 processes the images, and stores indications of the presence or absence of blood in the resultant data store 608. In some embodiments, the image analysis engine 616 may also cause presentation of an indication of the presence or absence of blood. In some embodiments, the image analysis engine 616 may cause a presentation of an overall indication of a presence or absence of blood based on a threshold number of pixels being determined to have one or more reflectance characteristics indicative of the presence of blood. In some embodiments, the image analysis engine 616 may cause a presentation of an overall indication of a presence or absence of blood based on whether a presence of blood is determined for more than a threshold number of pixels. In some embodiments, the presence of blood is determined for more than a threshold number of contiguous pixels. In some embodiments, the image analysis engine 616 may cause presentation of a false-color image that shows pixels that have been determined to have one or more reflectance characteristics indicative of the presence of blood. Because blood may be present in streaks on the surface of a bodily sample due to the physiology of how blood is introduced in the bodily sample, this false-color image may be particularly helpful in making a clinical determination of the presence or absence of blood and whether this presence or absence is indicative of a particular affliction.

As used herein, “engine” refers to logic embodied in hardware or software instructions, which can be written in one or more programming languages, including but not limited to C, C++, C#, COBOL, JAVA™, PHP, Perl, HTML, CSS, Javascript, VBScript, ASPX, Go, MATLAB™, and Python. An engine may be compiled into executable programs or written in interpreted programming languages. Software engines may be callable from other engines or from themselves. Generally, the engines described herein refer to logical modules that can be merged with other engines, or can be divided into sub-engines. The engines can be implemented by logic stored in any type of computer-readable medium or computer storage device and be stored on and executed by one or more general purpose computers, thus creating a special purpose computer configured to provide the engine or the functionality thereof. The engines can be implemented by logic programmed into an application-specific integrated circuit (ASIC), a field-programmable gate array (FPGA), or another hardware device.

As used herein, “data store” refers to any suitable device configured to store data for access by a computing device. One example of a data store is a highly reliable, high-speed relational database management system (DBMS) executing on one or more computing devices and accessible over a high-speed network. Another example of a data store is a key-value store. However, any other suitable storage technique and/or device capable of quickly and reliably providing the stored data in response to queries may be used, and the computing device may be accessible locally instead of over a network, or may be provided as a cloud-based service. A data store may also include data stored in an organized manner on a computer-readable storage medium, such as a hard disk drive, a flash memory, RAM, ROM, or any other type of computer-readable storage medium. One of ordinary skill in the art will recognize that separate data stores described herein may be combined into a single data store, and/or a single data store described herein may be separated into multiple data stores, without departing from the scope of the present disclosure.

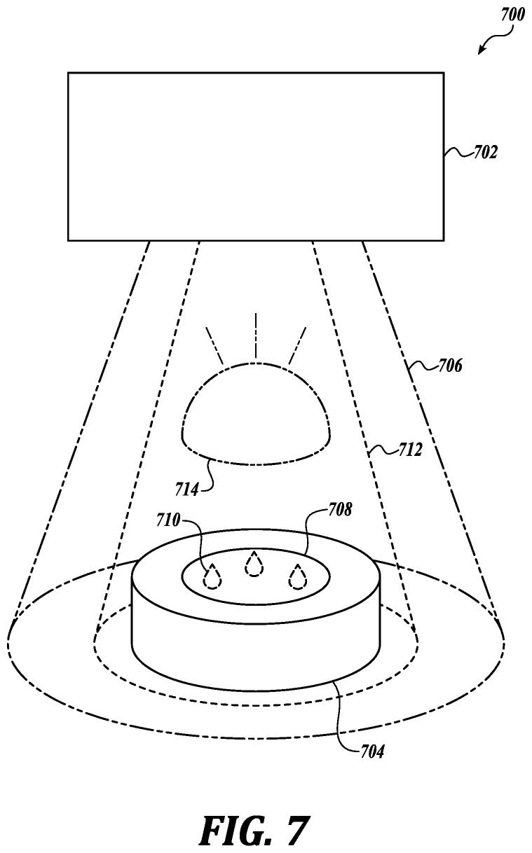

FIG. 7 illustrates a diagram of a non-limiting, example system for detecting a presence or an absence of blood in a bodily sample according to various aspects of the present disclosure. As shown, the system 700 includes a housing 702 having a light source and an image capturing device. The light source emits light within a field-of-illumination (FOI) 706 to illuminate a sample holder 704. [should describe sample holder 704 here].

The sample holder 704 has present a bodily sample 708. The light illuminates the bodily sample 708. As the light illuminates the bodily sample 708, the image-capturing device, having a field-of-view (FOV) 712, captures one or more images of the bodily sample 708 along with a section of the sample holder 704 present within the FOV 712. If any blood 710 is present in the bodily sample 708, the reflected light 714 reflected from the bodily sample 708 will have distinctive characteristics as discussed above, that are detectable in the one or more images captured by the image-capturing device.

In some embodiments, the image-capturing device may include the one or more cameras 610 of the bodily sample analysis computing system 600.

In some embodiments, the image-capturing device may include a photo-detecting sensor or device configured to detect the reflected light 714 reflected from the bodily sample 708.

In some embodiments, the housing 702 includes or otherwise comprises the systems and devices of the bodily sample analysis computing system 600.

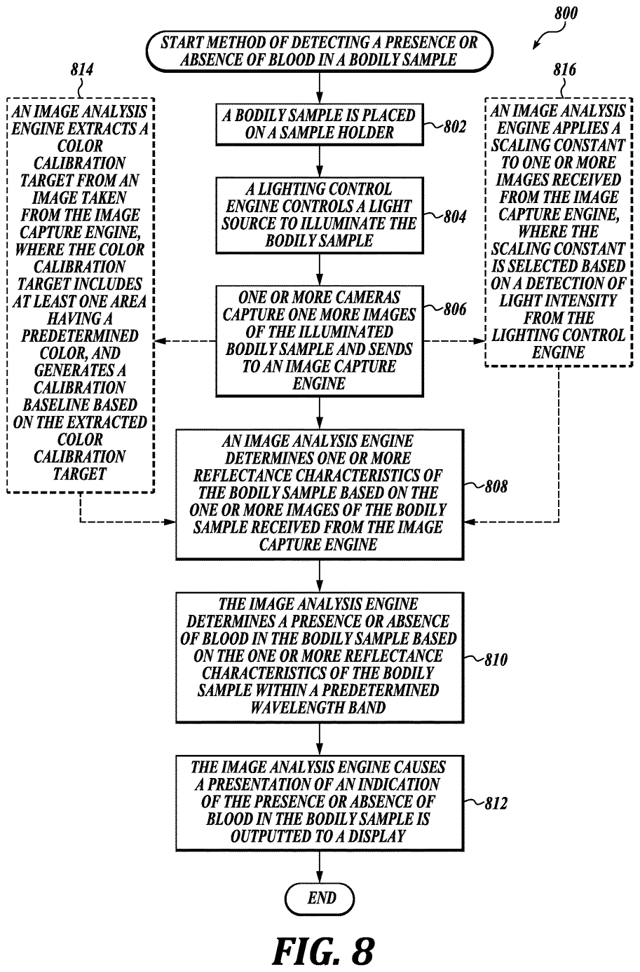

FIG. 8 illustrates a flowchart of a non-limiting, example method 800 of detecting a presence or an absence of blood in a bodily sample.

From a start block, the method 800 proceeds to block 802, where a bodily sample 708 is placed on a sample holder 704. In some embodiments, the bodily sample 708 is within the sample holder 704. In some embodiments, the bodily sample 708 is placed on a surface of the sample holder 704. In some embodiments, the bodily sample 708 is partially within the sample holder 704, with a portion of the bodily sample 708 exposed to the outside. In some embodiments, the sample holder 704 includes, but is not limited to, a toilet bowl, a urinal, a microscope slide, a petri dish, a vial, a beaker, a flask, or any partially exposed container or receptacle.

Proceeding to block 804, a lighting control engine 612 of a bodily sample analysis computing system 600 controls a light source with a FOI 706 to illuminate the bodily sample 708. The lighting control engine 612 causes the light source to illuminate the bodily sample 708 enough for the bodily sample 708 to reflect the light back to the bodily sample analysis computing system 600. Proceeding to block 806, one or more cameras 610 capture one or more images of the illuminated bodily sample 708 and sends them to an image capture engine 614. In some embodiments, the one or more cameras 610 capture images of the bodily sample 708 along with the sample holder 704. In some embodiments, the one or more images include a spectral image, an RGB image, a multispectral image, and a hyperspectral image.

In some embodiments, each image of the plurality of images is illuminated with a different LED of a plurality LEDs of the light source. In some embodiments, the plurality of LEDs includes one or more first LEDs, one or more second LEDs, and one or more third LEDs. In some embodiments, the one or more first LEDs produce light at wavelengths below a predetermined wavelength band, the one or more second LEDs produce light at wavelengths above a predetermined wavelength band, and the one or more third LEDs produce light at wavelengths within a predetermined wavelength band. In some embodiments, the one or more first LEDs produce light at wavelengths less than 520 nm, the one or more second LEDs produce light at wavelengths greater than 620 nm, and the one or more third LEDs produce light at wavelengths between the wavelengths of the one or more first LEDs and the wavelengths of the one or more second LEDs.

In some embodiments, the image capture engine 614 associates a captured image with a wavelength of the LED used during capture. In some embodiments, each captured image of the one or more images is associated with an individual wavelength of an LED used during capture. In some embodiments, artifacts and/or noise are captured by the one or more cameras 610 and are sent to the image capture engine 614.

In some embodiments, block 806 includes the actions of optional block 814, where the image analysis engine 616 generates a calibration baseline. In some embodiments, the image analysis engine 616 extracts a color calibration target from an image received from the image capture engine 614, where the color calibration target includes at least one area having a predetermined color. In some embodiments, the predetermined color is selected by a user. In some embodiments, the predetermined color includes a portion of the background of the sample holder 704 that has no bodily sample and is of a known color. One non-limiting example of such a background is a white toilet bowl, though other backgrounds with similar characteristics may be used in other embodiments. In some embodiments, the predetermined color includes or otherwise comprises a color selected from a color reference card visible in the captured image (e.g., a ColorChecker chart by X-Rite). The image analysis engine 616 may then generate the calibration baseline based on the extracted color calibration target.

In some embodiments, block 806 includes the actions of optional block 816, where the image analysis engine 616 applies a scaling constant. In some embodiments, the image analysis engine 616 applies a scaling constant to one or more images received from the image capture engine 614. In some embodiments, the scaling constant includes or otherwise comprises a reflectance standard, a brightness boost, or an RGB normalization. In some embodiments, the scaling constant is selected based on a detection of light intensity from the lighting control engine 612. The scaling constant K accounts for the intensity of light from the bodily sample, but does not affect the shape of the spectrum. A calibration reflectance spectrum may be used to adjust for wavelength dependence of the light source and for the camera detector efficiency. Put another way, the intensity of reflectance R affects the calculated optical density OD, so the technique may use the background color of the bodily sample as a reference to specify K, but if the OD_{green}>OD_{ref.green} based on side wavelengths, then blood will have been detected.

Proceeding to block 808, an image analysis engine 616 determines one or more reflectance characteristics R of the bodily sample 708 based on the one or more images of the bodily sample 708 received from the image capture engine 614. In some embodiments, a reflectance characteristic R of the one or more reflectance characteristics R includes or otherwise comprises an optical density OD. In some embodiments, the image analysis engine 616 determines the optical density OD by utilizing an operation based on one of the Expressions (1) or (8) to convert the reflectance characteristic R into the optical density OD. In some embodiments, the operation to convert the reflectance characteristic R into the optical density OD includes taking into account the blood content labeled by B, as well as the constants a and K, which correspond to each wavelength, as determined by Expression (10).

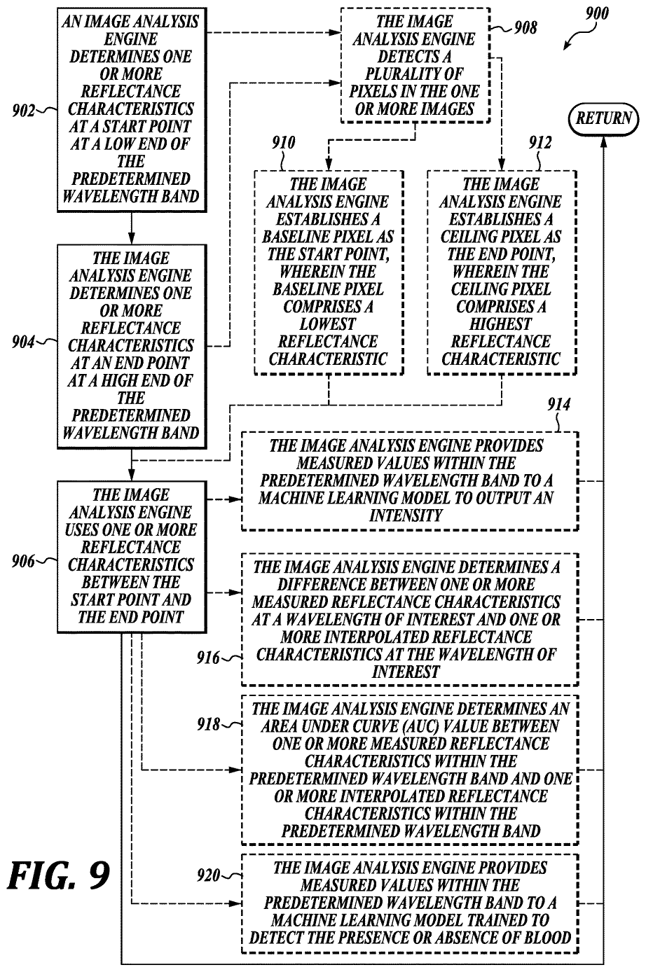

Proceeding to block 810, the image analysis engine 616 determines a presence or absence of blood in the bodily sample 708 based on the one or more reflectance characteristics of the bodily sample 708 within a predetermined wavelength band. In some embodiments, the predetermined wavelength band includes or otherwise comprises a wavelength range that is preselected by the bodily sample analysis computing system 600. In some embodiments, the bodily sample analysis computing system 600 selects the predetermined wavelength band based on factors such as: image quality, device specifications, background, multiple images, pixels, and measured values or reflectance. In some embodiments, the predetermined wavelength band includes or otherwise comprises a range that is a standard deviation of a wavelength range in which absorption by blood occurs. In some embodiments, the predetermined wavelength band is 530 nm-600 nm. Any suitable procedure may be used at block 810 to determine the presence or absence of blood. A non-limiting example embodiment of a suitable procedure is illustrated in FIG. 9 and discussed in further detail below.

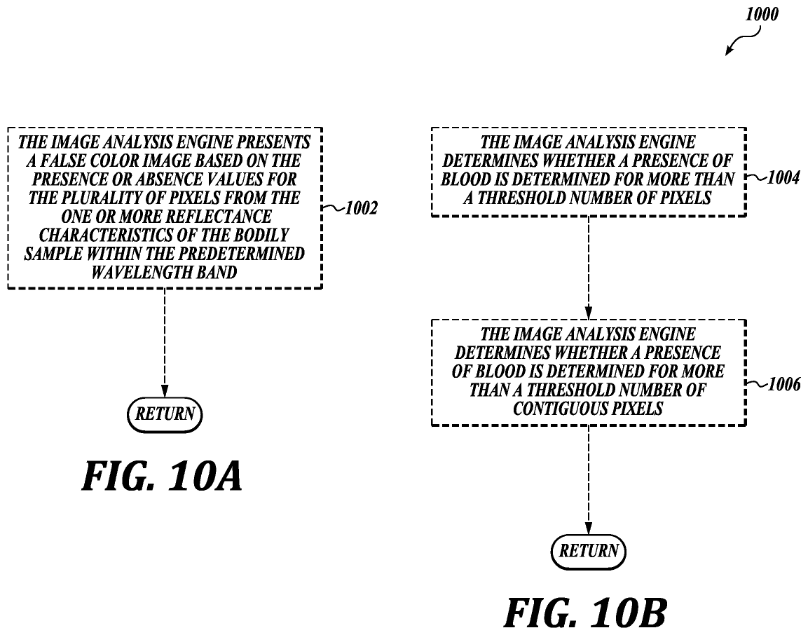

The method 800 then proceeds to block 812. At block 812, in some embodiments, the image analysis engine 616 causes a presentation of an indication of the presence of blood to be outputted to a display. In some embodiments, the display includes or otherwise comprises a display on a device of the bodily sample analysis computing system 600. Any suitable technique may be used to generate the presentation of the indication of the presence of blood, including but not limited to the non-limiting examples illustrated in FIG. 10 and described in further detail below. Upon completion of block 812, the method 800 proceeds to the End block and terminates.

FIG. 9 illustrates a non-limiting example of a procedure 900 for determining the presence or absence of blood in an image according to various aspects of the present disclosure. The procedure 900 is an example of a procedure suitable for use at block 810 of the method 800 described above.

The procedure 900 advances to block 902. Here, the image analysis engine 616 determines one or more reflectance characteristics at a start point at a low end of the predetermined wavelength band. Proceeding to block 904, the image analysis engine 616 determines one or more reflectance characteristics at an end point at a high end of the predetermined wavelength band.

In some embodiments, the start point and the end point may be based on predetermined values. For example: In some embodiments, the low end of the predetermined wavelength band is in a range of 450-550 nm. In some embodiments, the low end of the predetermined wavelength band is 500 nm. In some embodiments, the high end of the predetermined wavelength band is in a range of 575-675 nm. In some embodiments, the high end of the predetermined wavelength band is 630 nm.

In other embodiments, the start point and the end point may be determined dynamically based on the content of the image. Such embodiments are illustrated as optional blocks 908-912. At optional block 908, the image analysis engine 616 detects a plurality of pixels in the one or more images. At optional block 910, the image analysis engine 616 establishes a baseline pixel as the start point, wherein the baseline pixel comprises a pixel having a lowest reflectance characteristic. In some embodiments, this baseline pixel corresponds to a reflectance characteristic of a bodily sample 708 with no blood present, or an empty background. At optional block 912, the image analysis engine 616 establishes a ceiling pixel as the end point, wherein the ceiling pixel comprises a highest reflectance characteristic. In some embodiments, the ceiling pixel corresponds to a reflectance characteristic of a bodily sample 708 in which saturation in absorption of light by the bodily sample 708 occurs. The start point and the end point are then used for further processing.

Proceeding to block 906, the image analysis engine 616 uses one or more reflectance characteristics between the start point and the end point to determine the presence or absence of blood. In some embodiments, the image analysis engine 616 reviews the one or more reflectance characteristics measured within the predetermined wavelength band and assesses them in view of the one or more reflectance characteristics measured each at the start point and the end point. In some embodiments, the image analysis engine 616 may use one or more of the techniques illustrated in blocks 914-920.

At block 914, the image analysis engine 616 provides measured values within the predetermined wavelength band to a machine learning model that is configured to output an intensity. In some embodiments, the measured values includes or otherwise comprises measured reflectance characteristics. In some embodiments, the measured values includes or otherwise comprises measured RGB values. In some embodiments, the measured values includes or otherwise comprises measured pixels. In some embodiments, the machine learning model comprises at least one of: a linear regression model, a logistic regression model, a naïve Bayes model, a decision tree model, a random forest model, a k-nearest neighbor (KNN) model, a k-means model, a support vector machine (SVM) model, an apriori model, a gradient boosting model, or an artificial neural network model.

At block 916, the image analysis engine 616 determines a difference between one or more measured reflectance characteristics at a wavelength of interest and one or more interpolated reflectance characteristics at the wavelength of interest. In some embodiments, the wavelength of interest includes or otherwise comprises a range of 550 nm to 600 nm. In some embodiments, the wavelength of interest includes or otherwise comprises 577 nm or 585 nm.

At block 918, the image analysis engine 616 determines an area under curve (AUC) value between one or more measured reflectance characteristics within the predetermined wavelength band and one or more interpolated reflectance characteristics within the predetermined wavelength band. In some embodiments, the one or more interpolated reflectance characteristics within the predetermined wavelength band is determined by straight-line interpolation. In some embodiments, using a similar approach described in FIGS. 3A-3B, a straight-line interpolation is used to determine one or more interpolated reflectance characteristics, which is then used in conjunction with one or more measured reflectance characteristics to determine an AUC value.

At block 920, the image analysis engine 616 provides measured values within the predetermined wavelength band to a machine learning model trained to detect the presence or absence of blood. As opposed to the machine learning model described in block 914 which generated predictions of intensity, the machine learning model of block 920 directly outputs a prediction of the presence or absence of blood. In some embodiments, the machine learning model comprises at least one of: a linear regression model, a logistic regression model, a naïve Bayes model, a decision tree model, a random forest model, a k-nearest neighbor (KNN) model, a k-means model, a support vector machine (SVM) model, an apriori model, a gradient boosting model, or an artificial neural network model.

The procedure 900 then advances to an end terminal and returns the determined presence or absence of blood to its caller. In some embodiments, the procedure 900 may return a single presence or absence value for an entire image. In some embodiments, the procedure 900 may determine multiple presence or absence values. For example, the procedure 900 may execute block 902 and block 904 (with optional blocks 908, 910, and 912) once for an image in order to establish the reflectance characteristics at the start point and the end point, and then may execute the actions of block 906 separately for one or more pixels in the image in order to obtain per-pixel predictions of the presence or absence of blood.

FIG. 10A and FIG. 10B illustrate non-limiting examples of procedures for generating a presentation of an indication of a presence or absence of blood in a bodily sample according to various aspects of the present disclosure. The procedures are non-limiting examples of procedures suitable for use at block 812 of the method 800 described above.

In FIG. 10A, the procedure 1000 advances to block 1002, where the image analysis engine 616 presents a false-color image based on the presence or absence values for the plurality of pixels from the one or more reflectance characteristics of the bodily sample within the predetermined wavelength band. Upon completion of block 1002, the procedure 1000 proceeds to returns control to its caller. In some embodiments, the false-color image includes one or more images in one or more colors that differ from the one or more images retrieved by the image capture engine 614. For example, pixels of the false-color image that are associated with the absence of blood may be presented in the original color of the captured image, and pixels of the false-color image that are associated with the presence of blood may be presented in a different, high contrast color, such as bright green, red, or another suitable color. As another example, pixels of the false-color image that are associated with the absence of blood may be presented in black, white, or another solid color, and pixels of the false-color image that are associated with the presence of blood may be presented in a different solid color. By presenting the determined values in a false color image, the presence and pattern of blood detected in the bodily sample 708 is made immediately apparent to a reviewer.

In FIG. 10B, the procedure 1050 proceeds to block 1004, where the image analysis engine 616 determines whether a presence of blood is determined for more than a threshold number of pixels. In some embodiments, the threshold number of pixels may be a count or ratio of pixels in the entire image that are determined to depict a presence of blood. In some embodiments, the threshold number of pixels may be a threshold number of contiguous pixels that are determined to depict a presence of blood, in order to find an area of at least a minimum size. In some embodiments, the procedure 1050 may use both thresholds, or a different threshold. The presentation may then include an indication of whether the threshold was met or was not met (i.e., an indication of whether a threshold amount of blood was found or not). The procedure 1050 then advances and returns control to its caller.

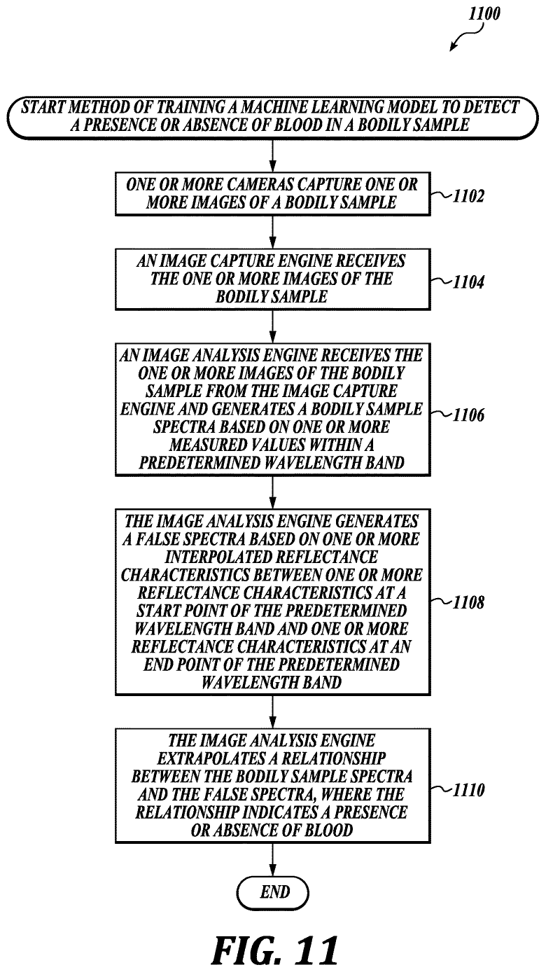

FIG. 11 illustrates a block diagram of a non-limiting, example method 1100 of training a machine learning model to detect a presence or absence of blood in a bodily sample.

From a start block, the method 1100 proceeds to block 1102, where one or more cameras 610 capture one or more images of a bodily sample 708.

In block 1104, the image capture engine 614 receives the one or more images of the bodily sample 708 from the one or more cameras 610.

In block 1106, the image analysis engine 616 receives the one or more images of the bodily sample 708 from the image capture engine 614 and generates a bodily sample spectra based on one or more measured values within a predetermined wavelength band.

In block 1108, the image analysis engine 616 generates a false spectra based on one or more interpolated reflectance characteristics between one or more reflectance characteristics at a start point of the predetermined wavelength band and one or more reflectance characteristics at an end point of the predetermined wavelength band.

In block 1110, the image analysis engine 616 extrapolates a relationship between the bodily sample spectra and the false spectra, where the relationship indicates a presence or absence of blood. In some embodiments, the relationship includes or otherwise comprises a visualization of the presence or absence of blood, such as a graph, a Blood Score, or a graphic indicating a positive detection of blood or a negative detection of blood. Upon completion of block 1110, the method 1100 proceeds to the End block. In some embodiments, the trained machine model produced from performing the steps in method 1100 is used in performing the steps described in block 920 of procedure 900 described in FIG. 9.

The complete disclosure of all patents, patent applications, and publications, and electronically available material cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern.

In the foregoing detailed description, numerous specific details are set forth to provide a thorough understanding of various embodiments. One skilled in the relevant art will recognize, however, that the techniques described herein can be practiced without one or more of the specific details, or with other methods, components, materials, etc. In other instances, well-known structures, materials, or operations are not shown or described in detail to avoid obscuring certain aspects.

Reference throughout this specification to “some embodiments,” or “an embodiment,” means that a particular feature, structure, or characteristic described in connection with the embodiment is included in at least some embodiments. Thus, the appearances of the phrase “in some embodiments,” “in one aspect,” or “in an embodiment,” in various places throughout this specification are not necessarily all referring to the same embodiment. Furthermore, the particular features, structures, or characteristics may be combined in any suitable manner in some embodiments.

The words “exemplary” and/or “demonstrative” are used herein to mean serving as an example, instance, or illustration. For the avoidance of doubt, the subject matter disclosed herein is not limited by such examples. In addition, any aspect or design described herein as “exemplary” and/or “demonstrative” is not necessarily to be construed as preferred or advantageous over other aspects or designs, nor is it meant to preclude equivalent exemplary structures and techniques known to those of ordinary skill in the art. Furthermore, to the extent that the terms “includes,” “has,” “contains,” and other similar words are used in either the detailed description or the claims, such terms are intended to be inclusive—in a manner similar to the term “comprising” as an open transition word—without precluding any additional or other elements.

The foregoing detailed description and examples have been given for clarity of understanding only. No unnecessary limitations are to be understood therefrom. The disclosure is not limited to the exact details shown and described, for variations obvious to one skilled in the art will be included within the disclosure defined by the claims.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure.

Specific elements of any foregoing embodiments can be combined or substituted for elements in other embodiments. Moreover, the inclusion of specific elements in at least some of these embodiments may be optional, wherein further embodiments may include one or more embodiments that specifically exclude one or more of these specific elements. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, 10 when used in this application, shall refer to this application as a whole and not to any particular portions of the application.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present disclosure. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

All of the references cited herein are incorporated by reference. Aspects of the disclosure can be modified, if necessary, to employ the systems, functions, and concepts of the above references and application to provide yet further embodiments of the disclosure. These and other changes can be made to the disclosure in light of the detailed description.

It will be appreciated that, although specific embodiments of the disclosure have been described herein for purposes of illustration, various modifications may be made without deviating from the spirit and scope of the disclosure. Accordingly, the disclosure is not limited except as by the claims.

1. A computer-implemented method of detecting a presence or absence of blood in a bodily sample, the method comprising:
receiving, by a computing system, one or more images of the bodily sample;
determining, by the computing system, one or more reflectance characteristics of the bodily sample based on the one or more images of the bodily sample;
determining, by the computing system, a presence or absence of blood in the bodily sample based on the one or more reflectance characteristics of the bodily sample within a predetermined wavelength band; and
causing, by the computing system, an indication of the presence or absence of blood to be presented on a display device.

2-3. (canceled)

4. The computer-implemented method of claim 1, wherein the bodily sample comprises a sputum, urine, or stool.

5. The computer-implemented method of claim 1, wherein a reflectance characteristic of the one or more reflectance characteristics comprises an optical density.

6. The computer-implemented method of claim 1, the method further comprising scaling, by the computing system, the received one or more images of the bodily sample, wherein the scaling comprises:
applying, by the computing system, a scaling constant to the received one or more images, wherein the scaling constant is selected based on a detection of light intensity.

7. The computer-implemented method of claim 1, wherein the determining the presence or absence of blood in the bodily sample based on the one or more reflectance characteristics of the bodily sample within the predetermined wavelength band includes:
determining one or more reflectance characteristics at a start point at a low end of the predetermined wavelength band;
determining one or more reflectance characteristics at an end point at a high end of the predetermined wavelength band; and
comparing one or more reflectance characteristics within the predetermined wavelength band to one or more interpolated reflectance characteristics between the one or more reflectance characteristics at the start point and the one or more reflectance characteristics at the end point.

8. The computer-implemented method of claim 7, wherein the determining one or more reflectance characteristics at the start point at the low end of the predetermined wavelength band comprises:
detecting a plurality of pixels in the one or more images; and
establishing a baseline pixel as the start point, wherein the baseline pixel comprises a lowest reflectance characteristic.

9. The computer-implemented method of claim 7, wherein the determining one or more reflectance characteristics at an end point at a high end of the predetermined wavelength band comprises:
detecting a plurality of pixels in the one or more images; and
establishing a ceiling pixel as the end point, wherein the ceiling pixel comprises a highest reflectance characteristic.

10. The computer-implemented method of claim 7, wherein the comparing the one or more reflectance characteristics within the predetermined wavelength band to the one or more interpolated reflectance characteristics between the one or more reflectance characteristics at the start point and the one or more reflectance characteristics at the end point includes:
providing measured values within the predetermined wavelength band to a machine learning model; and
using an intensity output by the machine learning model as the indication of the presence or absence of blood.

11. The computer-implemented method of claim 10, wherein the machine learning model comprises at least one of: a linear regression model, a logistic regression model, a naïve Bayes model, a decision tree model, a random forest model, a k-nearest neighbor (KNN) model, a k-means model, a support vector machine (SVM) model, an apriori model, a gradient boosting model, or an artificial neural network model.

12-15. (canceled)

16. The computer-implemented method of claim 14, wherein the one or more images of the stool are received from a camera of a smartphone, a hyperspectral camera, a multispectral camera, or a spectrometer.

17-19. (canceled)

20. The computer-implemented method of claim 7, wherein the low end of the predetermined wavelength band is in a range of 450-550 nm, and wherein the high end of the predetermined wavelength band is in a range of 575-675 nm.

21. The computer-implemented method of claim 20, wherein the low end of the predetermined wavelength band is 500 nm, and wherein the high end of the predetermined wavelength band is 630 nm.

22. The computer-implemented method of claim 7, wherein the comparing the one or more reflectance characteristics within the predetermined wavelength band to the one or more interpolated reflectance characteristics between the one or more reflectance characteristics at the start point and the one or more reflectance characteristics at the end point includes:
determining a difference between one or more measured reflectance characteristics at a wavelength of interest and the one or more interpolated reflectance characteristics at the wavelength of interest.

23. The computer-implemented method of claim 22, wherein the wavelength of interest is in a range of 550 nm to 600 nm.

24. The computer-implemented method of claim 23, wherein the wavelength of interest is 577 nm or 585 nm.

25. The computer-implemented method of claim 7, wherein the comparing the one or more reflectance characteristics within the predetermined wavelength band to the one or more interpolated reflectance characteristics between the one or more reflectance characteristics at the start point and the one or more reflectance characteristics at the end point includes:
determining an area under curve (AUC) value between one or more measured reflectance characteristics within the predetermined wavelength band and the one or more interpolated reflectance characteristics within the predetermined wavelength band.

26. (canceled)

27. The computer-implemented method of claim 1, wherein the determining the presence or absence of blood in the stool based on the one or more reflectance characteristics of the stool within the predetermined wavelength band includes:
determining a presence or absence values for a plurality of pixels of the one or more images.

28. The computer-implemented method of claim 27, wherein the causing the indication of the presence or absence of blood to be presented on the display device includes presenting a false-color image based on the presence or absence values for the plurality of pixels.

29. The computer-implemented method of claim 27, wherein the causing the indication of the presence or absence of blood to be presented on the display device includes determining whether a presence of blood is determined for more than a threshold number of pixels.

30. The computer-implemented method of claim 29, wherein the determining whether a presence of blood is determined for more than a threshold number of pixels includes determining whether a presence of blood is determined for more than a threshold number of contiguous pixels.

31-35. (canceled)
